(19) United States
(12) Patent Application Publication
(10) Pub. No.: US 2003/0143518 A1
(43) Pub. Date: Jul. 31, 2003

(54) Method of drying blood plasma

(75) Inventors: Thomas Luck, Muenchen (DE); Armin Prasch, Muenchen (DE); Andreas Maurer, Freising (DE); Reinhard Nowak, Binzen (DE); Bernard Luy, Freiburg (DE)

Correspondence Address: Marshall & Melhorn, L.L.C., D. Edward Dolgorukov, Eighth Floor, Four SeaGate, Toledo, OH, 43604, US

(73) Assignee: Fraunhofer-Gesellschaft zur Foerderung der angewandten Forschung e.V.

(21) Appl. No.: 10/313,812

(22) Filed: Dec. 6, 2002

Related U.S. Application Data
(63) Continuation-in-part of application No. 08/836,587, filed on Aug. 20, 1997, now abandoned.
Application No. 08/836,587 is a 371 of international application No. PCT/DE95/01619, filed on Nov. 17, 1995.

(30) Foreign Application Priority Data
Nov. 18, 1994 (DE) P4441 167.7

(51) Int. Cl. A01N 1/02

(52) U.S. Cl. 435/2

(57) ABSTRACT

The invention relates to a method of drying blood plasma, blood plasma fractions or blood plasma products (material for treatment) obtained therefrom, the product being sprayed in a liquid or dissolved condition into an evacuable container, drying to the granular form being carried out by means of a fluidizing gas in the fluidized layer.

### RELATED APPLICATION

This Application is continuation-in-part of U.S. Application Ser. No. 08/836,587, filed Aug. 20, 1997, which is a filing under 35 USC 371 of PCT International Application No. PCT/DE95/01619 filed Nov. 17, 1995, which claims priority of German patent Application No. P44 41 167.7 filed Nov. 18, 1994, the entirety of which is incorporated herein by reference.

### FIELD OF THE INVENTION

The invention relates to a method of drying blood plasma, blood plasma fractions or products obtained therefrom by means of a fluidized bed process, and to a corresponding device for carrying out the method.

### BACKGROUND OF THE INVENTION

It is known that the treatment of patients with human blood plasma products, i.e., proteins and/or protein fractions, involves an extremely high risk of cross-infection by viruses, e.g., retroviruses or hepatitis viruses. In particular, patients suffering from haemophilia have an extremely high infection risk. Therefore, prior art has described various methods of virus inactivation or elimination. However, as described, all these processes influence the biological activity of the blood plasma product.

Most methods propose heat-inactivation. Thus, for example, the document (“Le fractionnement plasmatique. Progrès, Problèmes at Perspectives”. [Plasma Fractionation. Progress, Problems and Perspectives] Burnouf, T; Ann pharmaceutiques francaises; 1994, 52, No. 3, p. 124-136) describes a heat-inactivation of a liquid (at 60° C. over 10 hours) and lyophilised plasma products (nitrogen atmosphere, 60° C. over 10 hours). It is known from Patent No. EP 0 094 611 that dry, e.g., lyophilized, plasma protein preparations can be pasteurised with a small loss of activity. However, it is also known that such dry heating methods may result in extremely poor and irregular heat transfer, above all when large quantities are involved, which may lead to over-heating of areas of the preparations while at the same time having areas where heating is partially insufficient. This on the one hand leads to undesirably high damage to the plasma proteins, and on the other hand to the fact that total virus inactivation is not reliably ensured.

Chemical methods for inactivating viruses are also known from prior art. One such example is a solvent/detergent method for inactivating viruses which are surrounded by a lipid envelope that uses tri-e-butylphosphate with the addition of further detergent, such as Tween 80°. Another known chemical method involves the inactivation of alkylising substances, such as β-propiolaction, in combination with UV treatment Bundesanzeiger No. 161, 1994, “Bekanntmachungen über Maβnahmen zur Abwehr von Arzneimittelrisiken—Verhinderung des Risikos der Übertragung von hämatogenen Viren bei Arzneimitteln, die durch Fraktionierung aus plasma humanen Ursprungs hergestellt werden”.

[Announcements regarding Measures for Defence against Drug Risks—Preventing the Risk of Transfer of haemotogenic Viruses in Drugs produced by Fractionation of Plasma of Human Origin].

The methods described above all have the disadvantage of negatively influencing the biological activity known to be associated with particular blood plasma proteins of interest.

### SUMMARY OF THE INVENTION

There has been no lack of attempts to provide assistance here by means of so-called “gentle drying methods”. Such methods are lyophilisation and vacuum drying methods. The methods do in fact have the advantage of a gentle handling of the product, yet there is the disadvantage that the methods are extremely complex in terms of process technology and thus result in high investment and operational costs. A further unfavourable effect of these methods is the slow throughput and inflexible process configuration.

Proceeding from this point, it is therefore the object of the present invention to propose a method for gentle drying of blood plasma, blood plasma fractions or blood plasma products, in which there is no influence on the biological activity known to be associated with particular blood plasma proteins contained in the blood plasma, blood plasma fractions or blood plasma products. At the same time, this method is to be simple and cost-effective to carry out.

Thus, it is proposed to dry the blood plasma, blood plasma fractions or blood plasma products in a fluidized bed chamber. For this reason, the blood plasma, blood plasma fraction or blood plasma product to be dried is sprayed in a liquid form into an evacuable container (according to Patent No. EP-A-0-149-266B1) and dried in the fluidized bed by means of a fluidizing gas. The liquid may be passed either in counter-flow to the fluidizing gas (top-spray method) or in the same flow direction (bottom-spray method). The purpose of the fluidizing gas is not only to induce turbulence in the blood plasma, blood plasma fraction or blood plasma product, but also to provide convective heat transfer. As such, according to the invention, a heated gas is used as a fluidizing gas. By virtue of the fact that the liquid blood plasma, blood plasma fraction or blood plasma product is finely distributed in the evacuable container by an appropriate nozzle, optimum drying can be achieved by the fluidizing gas.

By measuring the product temperature during the fluidized bed process and formulating a process control pattern based thereon, drying which gently handles the product can be maintained. The temperature of the fluidizing gas is selected in accordance with the particular plasma protein or proteins of interest in the liquid blood plasma, blood plasma fraction or blood plasma product, such that the gentlest possible drying is brought about. The temperatures is referred to as the maximum acceptable temperature, or the highest temperature to which one or more particular plasma proteins may be exposed with no loss of the biological activity known to be associated with said proteins. For most relevant plasma proteins, such as albumin, thrombin and Factor XIII, the temperature can lie in a range from 15 to 75° C. It has been further determined that a more narrow range of 15-35° C., and most preferably between 20-28° C., may be used where fibrinogen is the plasma protein of interest. Experimental results regarding the determination of the temperature have been included below.

Either air or an inert gas, such as nitrogen as described by prior art, can be used as a fluidizing-gas. Drying is continued until the blood plasma, blood plasma fraction or blood plasma product is present in a finely distributed granular form. The granules produced by the inventive method are 100-200 μm in diameter, an extremely finely distributed form, so that optimum heat transfer from the fluidizing gas is guaranteed. Moisture content by weight, or total weight of the granules produced by the inventive method, is less than 5%.

The method is preferably carried out in such a way that the fluidizing gas is circulated through an evacuable container. When performed in this manner, the fluidizing gas also has the purpose of ensuring removal of the evaporated or dried liquid from the moist product. In addition, in order to recondition the fluidizing gas, a condenser is provided for dehumidification and a heat exchanger for heating.

In order to attain particularly gentle drying conditions, i.e., to perform drying of the liquid blood plasma, blood plasma fraction or blood plasma product at temperatures lower than those previously described, the drying can also be carried out at a reduced operational pressure. In this case the pressure can be reduced to below 500 mbars. In order to compensate for the disadvantage of less convective drying performance at reduced operational pressure, energy which is made available for drying can be supplied via an additional energy source, whose transfer mechanism is not tied to the convective heat transfer. For example, this can be carried out by microwave radiation which is introduced into the fluidized bed. In this way additional freedom is achieved in the control and execution of the drying process of the controlled heating.

It is further proposed that, an additional controlled heat treatment for inactivation of viruses is used beginning with the end of drying of the liquid blood plasma, blood plasma fraction or blood plasma product. Such heat treatment is used only after the drying has been almost completed, as the dried material in the form of the fine granular form described above is considerably more heat-stable than the liquid starting material. Therefore, according to the invention, heat treatment is applied only after drying or at the beginning of the last phase of the drying process. In all cases, a condition of this is that the material that is to receive additional heat treatment should already be present in a finely-distributed granular form. The operational conditions previously known to be necessary conventionally for inactivating viruses in the case of dried blood plasma products can be set for the heat treatment. The essential point is that inactivation takes place in the fluidized condition. This provides ideal heat transfer conditions, so that a uniform application of temperature and thus uniform inactivation is achieved. Inactivation can be carried out in air, or in an ozone-enriched air atmosphere. Experimental results for virus inactivation in dry albumin powder are included below.

In addition to the convective supply of energy through the fluidizing medium, heat can also be generated in a controlled manner in the product by means of an external energy source. This is more advantageously achieved by means of microwave radiation.

In addition, the fluidized product may be irradiated with UV light through a window of UV impermeable material provided on the periphery of the reactor in the area of the fluidized bed, in order to inactivate viruses. The products may also at the same time be directly irradiated by means of a UV radiator mounted in the reactor.

The method according to the invention also permits chemical inactivation. Chemical inactivation of viruses is provided according to the solvent/detergent method, in such a way that a corresponding solvent is added to the liquid product before it is sprayed into the evacuable chamber. These solvents are known from prior art, as previously described.

### EXAMPLE 1

### Detection of Biological Activity of Albumin in Granules Prepared from a Starting Material of Freeze-Dried Albumin Powder

Albumin activity was detected using the following high-phase liquid chromatography (HPLC) method.

Sample preparation:

Puffer eluent: 1 liter of 0.1 M Na₂SO₄ solution+1 liter of 0.1 M NaH₂PO₄ solution in purified water, final pH adjusted to 6.8 with NaOH.

Liquid samples: dissolved with Puffer eluent and filtered with 0.45 μm filter.

Solid samples: 20 mg of solid samples were dissolve in 20 ml Puffer eluent and filtered through a 0.45 μm filter to a protein concentration of 0.5-1.0 grams/liter.

HPLC Columns:

Pre-column: Bio-Sil SEC, Ref. No. 125-0073

Main column: Bio Rad Bio-Sil SEC 250, Ref. No. 125-0062.

Temperature: 30° C.

Flow: 0.8 ml/minute

Detector: Gyynkothek UVD 160-220 nm

Standard Bio Rad Ref. No. 151-1901

Conditions of fluidized bed processing:

Freeze-dried albumin powder with different moisture contents was processed in a fluid bed granulator (type Glatt GPCG 1.1). The following moisture contents of freeze-dried albumin powder were investigated:

Residual moisture content of the starting material (freeze-dried albumin powder) was 6.5%, 10% and 12% moisture content by weight, i.e., % w/w, as detected by IR method, 120° C. for 20 minutes.

Air volume for all experiments kept constant at 100 m³/hour.

Load of starting material for all batches kept constant at 600 grams per batch.

Product temperature kept constant at 60° C. or 75° C.

Albumin denaturation was measured as relative deviation of activity of the samples (A) as compared to activity of the starting material (A₀) as detected by the HPLC method.

As shown in FIGS. 1 and 2, the granulation of freeze-dried albumin with a residual moisture content below 6.5% w/w at product temperatures of 60° C. or 75° C. does not negatively affect the activity of albumin, which corresponds to preservation of the molecular structure of albumin. It furthermore can be assumed that temperature below 60° C. will also not negatively affect the activity of albumin.

### EXAMPLE 2

### Detection of Biological Activity of Fibrinogen in Granules Prepared from a Starting Material of Aqueous Solution of Fibrinogen

Fibrinogen granules are produced by spray granulation out of an aqueous solution of fibrinogen followed by a fluid bed drying/granulation.

Process conditions:

Equipment: Fluid bed (Glatt GPCG 1.1) with top-spray configuration.

Starting load of inert material: 300 grams

Air volume: 60-80 m³/hour

Spray rate: 3-8 grams/minute

Inlet air temperature: 30-32° C.

Materials:

Starting load: D-Mannit DAB, Ph.Eur., Merck Eurolab, 1.05980.9050

Aqueous solution of fibrinogen: 10% w/w and 5% w/w

Solid composition: ˜17% albumin of total protein, ˜30-90 units/ml of Factor XIII, ˜80% fibrinogen of total protein.

The quality of samples processed with fluid bed granulation was measured with analytical centrifugation and capillary gel electrophoresis. It is known that fibrinogen is present at several aggregated states, which are characteristic for fibrinogen formulations. Analytical centrifugation allows a direct calculation of the molecular weight of the sedimenting component from the observed sedimentation rate. An Analytical Ultracentrifuge Optima XL-A (Beckman) with detection cells with two-channels—Centerpieces with an optical wavelength of 1.2 cm at wavelength of 280 nm was used. Results showed that there is no difference in the distribution of the molecular weight between reference samples of lyophilized fibrinogen and fibrinogen granules processed with fluid bed granulation (see Table 1). Similar results were found by using size-exclusion chromatography to determine the molecular weight which correlates directly to the aggregation state of the fibrinogen formulation and accordingly to the activity of the fibrinogen.

### EXAMPLE 3

### Inactivation of Poliovirus in a Preparation of Dry Albumin

Poliovirus is an uncoated virus that is often used as a reference virus for research purposes. Experiments were performed with dry albumin powder prepared from an albumin solution contaminated with a known concentration of poliovirus. Virus concentration of the starting albumin solution was approximately 1×10⁸ virus/ml. The solution was then pre-dried in an exsicator at a temperature of 50° C. for 24 hours. Virus concentration after pre-drying was approximately 1×10⁵ virus/ml.

The moisture content of the dry albumin powder was 4% w/w, which is equal to 96% w/w of solid content. The dry, powdery, virus-contaminated albumin was then transferred in a fluid bed in order to carry out a defined virus inactivation step at comparably high temperatures. Within 15 minutes of fluid bed dry heat virus inactivation, the virus concentration could be reduced by 4-log steps. As shown by FIG. 3, the virus concentration of the product was with 10 viruses/ml below the detection limit of the analytical method for virus detection.

Process conditions:

Equipment: MiniGlatt fluid bed unit

Load of virus-contaminated dry albumin: 50 grams

Inlet air temperature: 100° C.

Air volume: 20 m³/hour

Further experiments were performed with the following parameters:

Equipment: MiniGlatt fluid bed unit

Load of virus-contaminated dry albumin: 50 grams

Inlet air temperature: 60° C. and 80° C.

Air volume: 20 m³/hour

UV radiation: 4 W/cm³

Results are summarized in Table 2. Note that according to the CPMP Note of Guidance on Virus Validation Studies: The Design, Contribution and Interpretaion of Studies Validating the Inactivation and Removal of Viruses, revised, CPMP/BWP/268/95, p. 16, as promulgated by the Committee for Proprietary Medicinal Products of the Food and Drug Administration, “[l]og reductions of the order of 4 logs or more are indicative of a clear effect with the particular test virus under investigation.” Note further that the Guidelines also specify that log₁₀ reduction would be one factor considered in judging the effectivity of virus inactivation.

### BRIEF DESCRIPTION OF THE DRAWINGS

Further details, advantages and preferences of the invention will become apparent from the following example, and with reference to FIG. 1, FIG. 2, and FIG. 3.

FIG. 1 shows schematically the preferred embodiement of a device for carrying out the inventive method, in which the fluidizing gas is circulated.

FIG. 2 compares the denaturation of albumin that is dried in a fluidized bed at a temperature of 60° C. into granules wherein the starting material had 6.5%, 10% and 12.5% residual moisture content by weight.

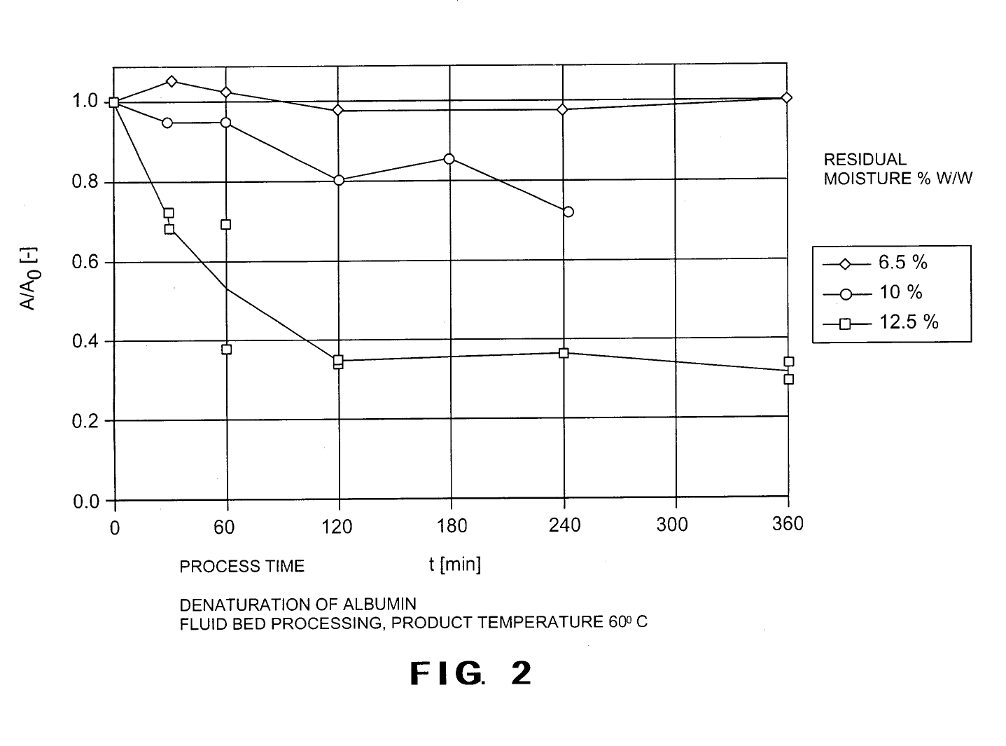

FIG. 3 compares the denaturation of albumin that is dried in a fluidized bed at a temperature of 75° C. into granules wherein the starting material had 6.5%, 10% and 12.5% residual moisture content by weight

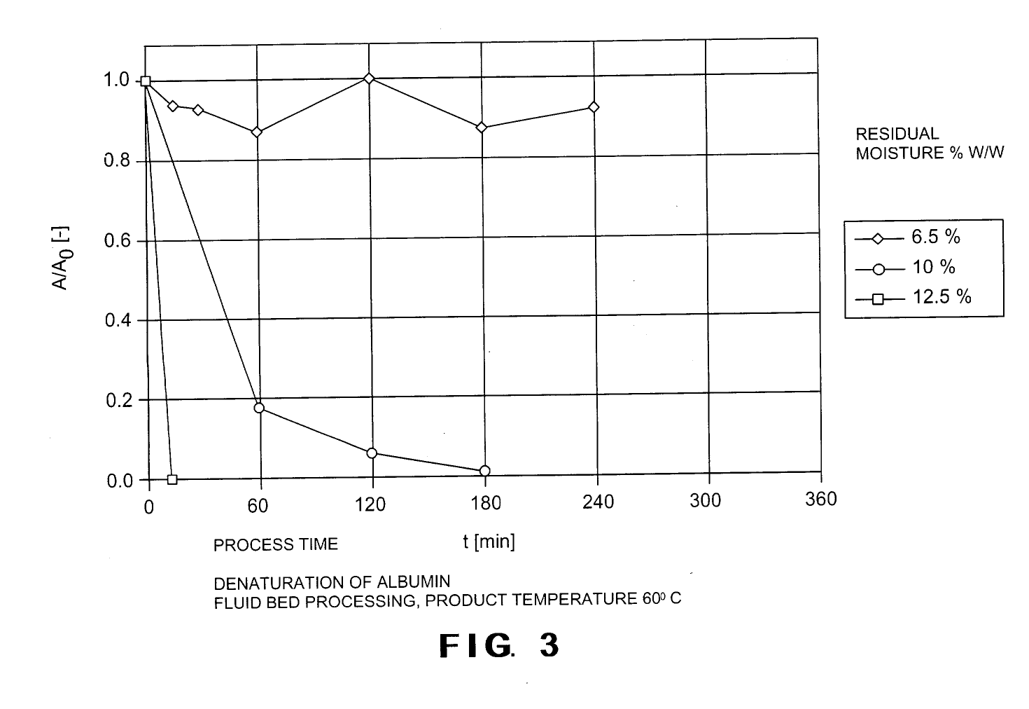

FIG. 4 shows the inactivation of poliovirus in a preparation of albumin dried in a fluidized bed into granules having 4% residual moisture content by weight with heat treatment at 100° C.

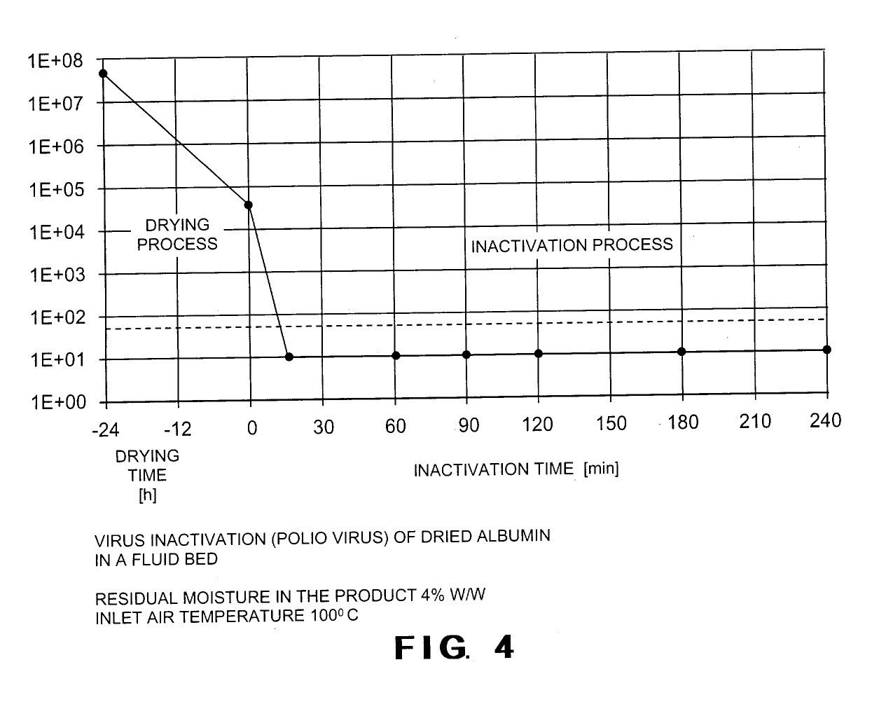

### DETAILED DESCRIPTION OF THE PREFERRED EMBODIMENT

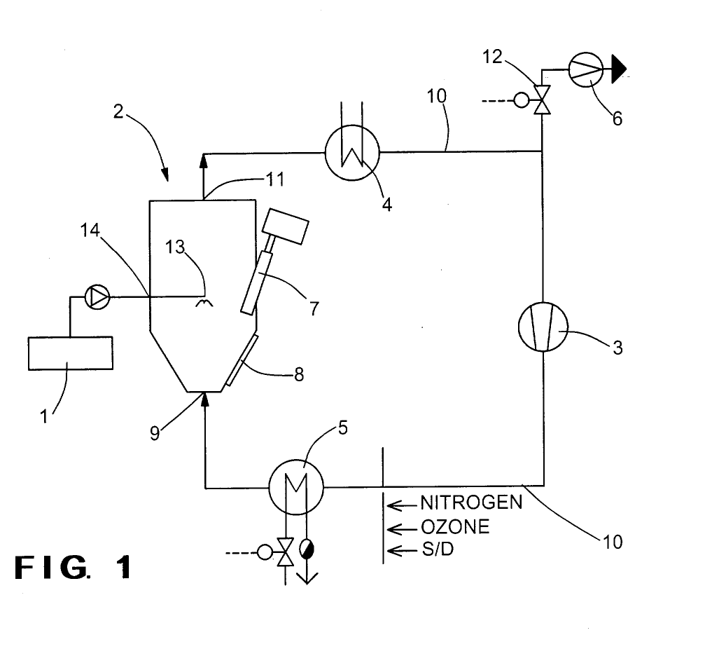

In the example, the container 2 is cylindrical in shape, and on the side at which the fluidizing gas is introduced, it is conical in form. The inlet 9 leads into the conical side of the eyacuable container 2, and inlet, in the example according to FIG. 1, being in the form of a hinged baffle base. At the same time there is incorporated in the periphery of the conical side of the container 2 a window 8, which is permeable to UV light. The window 8 is so designed that the product may also subsequently be additionally irradiated with UV light by means of an external light source (not shown). The intensity of the UV light can lie in the range of 1 to 2 m^{w}/cm². In addition, the container 2 has a microwave device 7 for reinforcing the drying and/or for subsequent heat treatment. For circulating the fluidizing gas there is provided a ring circuit 10, which leads from the outlet 11 to the inlet 9 of the container 2. For, reconditioning, a condenser 4 and a heat exchanger 5 are located in the ring circuit 10.

As a supplementary measure, ozone may likewise be introduced for inactivation. The fluidizing gas is circulated by a blower 3. For the case in which a reduced operational pressure must be set in the evacuable container 2, a corresponding vacuum pump 6 is provided, which can be disconnected from the ring circuit 10 by means of a valve 12. The liquid product is supplied by means of an antechamber 1 from which the product is passed through the inlet 14 to the spray head 13.

The method will be described in more detail in the following by means of an example. The example relates to the drying of human blood plasma with a device according to FIG. 1.

Freshly thawed blood plasma is sprayed at a temperature of about 0 to 4° C. through the nozzle 13, (two-component nozzle 9) into the empty fluidized bed chamber 2. The liquid may either be sprayed according to the top-spray method (see FIG. 1) from above onto the fluidized bed or, through a spray device attached at the top of the flow baffle base, from below in the same current direction as the carrier gas into the fluidized bed chamber (bottom-spray method).

The average fluidization speed comes to about 0.4 to 0.6 m/s.

The process is initiated preferably according to a method of DE 35 16 967 A1. During the following, actual fluidized bed drying, the operational pressure is reduced by the pump 6 to about 10 KPa. The temperature of introduced air lies at about 35° C. The moisture content of the introduced air or of the carrier gas should be less than 30%. By means of reducing the pressure to an operational pressure of 10 KPa, the boiling point of the liquid to be removed reduces (e.g. boiling temperature of water at 10 KPa is about 45° C.), and the cooling threshold or material surface temperature also drop (theoretically about 5° C.). In this way low drying temperatures gentle to the product (measured product temperatures lie at about 10 to 15° C.) can be produced. In this phase, drying is reinforced by microwave heating 7. The microwave system is coupled at a frequency of 2450 MHz and with a maximum power of 1.2 k^{w}. Irradiation by the microwave system is carried out in a way regulated by product temperature, i.e. the point in time and duration of the microwave irradiation and the maximum power applied are regulated in dependence on a fixed maximum acceptable product temperature.

Due to the known advantage of microwave applications which enable extremely fast process regulation by means of the inertia-free energy transfer, this form of energy supply is suitable to permit individual process guidance in drying and heating processes. The maximum acceptable product temperature is fixed at 25° C. By means of determining a corresponding kinetics, which produces a formal relationship between temperature loading, product water content and the reduction in the activity of active ingredient dependent thereon, the latter may be precisely determined. The possible spray rate under these conditions, in purely convective fluidized bed drying, comes to about 0.9 kg/h. If the drying performance is supplemented by microwave irradiation regulated by product temperature, drying can be carried out at a spray rate of about 1.9 kg/h. A constant water content of about 10 to 13% is set as an average product water content in the fluidized product. Drying is effected up to a product moisture content at which the active ingredient is sufficiently stable for further treatment, storage or distribution.

After, or towards the end of, the actual drying stage, heat treatment for inactivation of viruses is carried out in the same fluidized bed chamber 2. Fluidization of the dried product ensures the most uniform and effective possible heat transfer between carrier gas and the individual particles. Heating may for example be carried out at a pressure of 100 KPa and in an inert gas atmosphere (e.g. nitrogen) heated to 60° C. In dependence on the measured product temperature, the temperature of the inert gas atmosphere is regulated in such a way that a maximum temperature of 70° C. is not exceeded. Heating may be additionally supplemented by microwave radiation, and additional virus inactivation is possible by means of UV radiation.

The duration of heat treatment is based on the inactivation (titre reduction) to be achieved of possible viruses. Due to the ideal heat transfer conditions and the ideal uniformity of heating, clear overheating points or points of insufficient heating are not present, so that the duration of heating is clearly less than the conventional 10 hours.

1. A method of drying blood plasma, comprising drying liquid blood plasma, devoid of any binders, fillers and carriers, to a granular form in a bed of a heated fluidizing gas, monitoring the temperature of said blood plasma being dried so as not to exceed a temperature that has been determined as the maximum acceptable temperature, or the highest temperature to which a particular plasma protein may be exposed with no loss of the biological activity known to be associated with said protein.

2. A method of drying liquid blood plasma, consisting of:
a) providing a fluidized bed produced by a fluidizing gas;
b) directly spraying said blood plasma devoid of any binders, fillers and carriers, into an evacuable chamber containing said fluidizing bed, thereby introducing said. blood plasma into said fluidized bed;
c) drying said blood plasma to produce granules having a diameter of 100-200 μm, by raising the temperature of said blood plasma in said fluidized bed produced by said fluidizing gas; and
d) further heating said granules for a period of time and to a temperature higher than that of step c), which is high enough to inactivate viruses but does not exceed the fixed maximum acceptable product temperature, while said dried blood plasma is still in said fluidized bed produced by said fluidizing gas.

3. A method according to claim 2, wherein air or an inert gas is used as a fluidizing gas.

4. A method according to claim 2, wherein said further heating of said blood plasma in the fluidized condition to inactivate viruses is carried out at the beginning of the termination of drying step c).

5. A method according to claim 4, wherein the heat to inactivate viruses is applied to the dried blood plasma in the form of UV light or microwaves, or by heating said fluidizing gas.

6. A method according to claim 2, wherein a solvent is added to said blood plasma before it is introduced into said bed of said fluidizing gas to chemically inactivate viruses, thereby eliminating the need for step d).

7. A method according to claim 2, wherein said fluidizing gas is circulated and reconditioned wherein said reconditioning of said fluidizing gas carried out by means of a condenser for dehumidification and of a heat exchanger for heating.

8. A method of drying liquid blood plasma, consisting of:
a) providing a fluidized bed produced by a fluidizing gas;
b) directly spraying said blood plasma devoid of any binders, fillers and carriers, into an evacuable chamber containing said fluidizing bed, thereby introducing said blood plasma into said fluidized bed;
c) drying said blood plasma to produce granules having a diameter of 100-200 μm by reducing the pressure within the evacuable container below 500 mbars, raising the temperature of said blood plasma in said fluidized bed produced by said fluidizing gas, and supplying an additional energy source, the heat transfer mechanism of which is not convective in nature, such as microwave radiation; and
d) further heating said granules for a period of time and to a temperature higher than that of step c), which is high enough to inactivate viruses but does not exceed the fixed maximum acceptable product temperature, while said dried blood plasma is still in said fluidized bed produced by said fluidizing gas.

9. A method according to claim 8, wherein air or an inert gas is used as a fluidizing gas.

10. A method according to claim 8, wherein said further heating of said blood plasma in the fluidized condition to inactivate viruses is carried out at the beginning of the termination of drying step c).

11. A method according to claim 10 wherein the heat to inactivate viruses is applied to the dried blood plasma in the form of UV light or microwave, or by heating said fluidizing gas.

12. A method according to claim 8, wherein a solvent is added to said blood plasma before it is introduced into said bed of said fluidizing gas to chemically inactivate viruses, thereby eliminating the need for step d).

13. A method according to claim 8, wherein said fluidizing gas is circulated and reconditioned wherein said reconditioning of said fluidizing gas is carried out by means of a condenser for dehumidification and of a heat exchanger for heating.

14. A method of drying liquid blood plasma fractions obtained by separating said fractions from liquid blood plasma, comprising drying said blood plasma fractions, devoid of any binders, fillers and carriers, to a granular form in a bed of a heated fluidizing gas, monitoring the temperature of said blood plasma fractions being dried so as not to exceed a temperature that has been determined as the maximum acceptable temperature, or the highest temperature to which a particular plasma protein known to be present in the blood plasma factions may be exposed with no loss of the biological activity known to be associated with the protein.

15. A method of drying liquid blood plasma fractions, consisting of:
a) providing a fluidized bed produced by a fluidizing gas;
b) directly spraying said blood plasma fractions devoid of any binders, fillers and carriers, into an evacuable chamber containing said fluidizing bed, thereby introducing said blood plasma fractions into said fluidized bed;
c) drying said blood plasma fractions to p produce granules having a diameter of 100-200 μm, by raising the temperature of said blood plasma fractions in said fluidized bed produced by said fluidizing gas; and
d) further heating said granules for a period of time and to a temperature higher than that of step c), which is high enough to inactivate viruses but does not exceed the fixed maximum acceptable product temperature, while said dried blood plasma fractions are still in said fluidized bed produced by said fluidizing gas.

16. A method according to claim 15, wherein air or an inert gas is used as a fluidizing gas.

17. A method according to claim 15, wherein said further heating of said blood plasma fractions in the fluidized condition to inactivate viruses is carried out at the beginning of the termination of drying step c).

18. A method according to claim 17 wherein the heat to inactivate viruses is applied to the dried blood plasma fractions in the form of UV light or microwaves, or by heating said fluidizing gas.

19. A method according to claim 15, wherein a solvent is added to said blood plasma before it is introduced into said bed of said fluidizing gas to chemically inactivate viruses, thereby eliminating a need for step d).

20. A method according to claim 15, wherein said fluidizing gas is circulated and reconditioned wherein said reconditioning of said fluidizing gas is carried out by means of a condenser for dehumidification and of a heat exchanger for heating.

21. A method of drying liquid blood plasma fractions, consisting of:
a) providing a fluidized bed produced by a fluidizing gas;
b) directly spraying said blood plasma fractions devoid of any binders, fillers and carriers, into an evacuable chamber containing said fluidizing bed, thereby introducing said blood plasma fractions into said fluidized bed;
c) drying said blood plasma fractions to produce granules having a diameter of 100-200 μm by reducing the pressure within the evacuable container below 500 mbars, raising the temperature of said blood plasma fractions in said fluidized bed produced by said fluidizing gas, and supplying an additional energy source, the heat transfer mechanism of which is not convective in nature, such as microwave radiation; and
d) further heating said granules for a period of time and to a temperature higher than that of step c), which is high enough to inactivate viruses but does not exceed the fixed maximum acceptable product temperature, while said dried blood plasma fractions are still in said fluidized bed produced by said fluidizing gas.

22. A method according to claim 21, wherein air or an inert gas is used as a fluidizing gas.

23. A method according to claim 21, wherein said further heating of said blood plasma fractions in the fluidized condition to inactivate viruses is carried out at the beginning of the termination of drying step c).

24. A method according to claim 23, wherein the heat to inactivate viruses is applied to the dried blood plasma fractions in the form of UV light or microwaves, or by heating said fluidizing gas.

25. A method according to claim 21, wherein a solvent is added to said blood plasma fractions before said fractions are introduced into said bed of said fluidizing gas to chemically inactivate viruses, thereby eliminating a need for step d).

26. A method according to claim 21, wherein said fluidizing gas is circulated and reconditioned wherein said reconditioning of said fluidizing gas is carried out by means of a condenser for dehumidification and of a heat exchanger for heating.

27. A method of drying liquid blood plasma products, obtained by purifying said products from liquid blood plasma fractions, comprising drying said blood plasma products, devoid of any binders, fillers and carriers, to a granular form in a bed of a heated fluidizing gas, monitoring the temperature of said blood plasma products being dried so as not to exceed a temperature that has been determined as the maximum acceptable temperature, or the highest temperature to which a particular protein known to be present in the blood plasma products may be exposed with no loss of the biological activity known to be associated with said protein.

28. A method of drying liquid blood plasma products, consisting of:
a) providing a fluidized bed produced by a fluidizing gas;
b) directly spraying said blood plasma products devoid of any binders, fillers and carriers, into an evacuable chamber containing said fluidizing bed, thereby introducing said blood plasma products into said fluidized bed;
c) drying said blood plasma products to produce granules having a diameter of 100-200 μm, by raising the temperature of said blood plasma in said fluidized bed produced by said fluidizing gas; and
d) further heating said granules for a period o of time and to a temperature higher than that of step c), which is high enough to inactivate viruses but does not exceed the fixed maximum acceptable product temperature, while said dried blood plasma products are still in said fluidized bed produced by said fluidizing gas.

29. A method according to claim 28, wherein air or an inert gas is used as a fluidizing gas.

30. A method according to claim 28, wherein said further heating of said blood plasma products in the fluidized condition to inactivate viruses is carried out at the beginning of the termination of drying step c).

31. A method according to claim 30, wherein the heat to inactivate viruses is applied to the dried blood plasma products in the form of UV light or microwave, or by heating said fluidizing gas.

32. A method according to claim 28, wherein a solvent is added to said blood plasma fractions before said fractions are introduced into said bed of said fluidizing gas to chemically inactivate viruses, thereby eliminating a need for step d).

33. A method according to claim 28, wherein said fluidizing gas is circulated and reconditioned wherein said reconditioning of said fluidizing gas is carried out by means of a condenser for dehumidification and of a heat exchanger for heating.

34. A method of drying liquid blood plasma products, consisting of:
a) providing a fluidized bed produced by a fluidizing gas;
b) directly spraying said blood plasma products devoid of any binders, fillers and carriers, into an evacuable chamber containing said fluidizing bed, thereby introducing said blood plasma products into said fluidized bed;
c) drying said blood plasma products to produce granules having a diameter of 100-200 μm by reducing the pressure within the evacuable container below 500 mbars, raising the temperature of said blood plasma products in said fluidized bed produced by said fluidizing gas, and supplying an additional energy source, the heat transfer mechanism of which is not convective in nature, such as microwave radiation; and
d) further heating said granules for a period of time and to a temperature higher than that of step c), which is high enough to inactivate viruses but does not exceed the fixed maximum acceptable product temperature, while said dried blood plasma products are still in said fluidized bed produced by said fluidizing gas.

35. A method according to claim 34, wherein air or an inert gas is used as a fluidizing gas.

36. A method according to claim 34, wherein said further heating of said blood plasma products in the fluidized condition to inactivate viruses is carried out at the beginning of the termination of drying step c).

37. A method according to claim 36, wherein the heat to inactivate viruses is applied to the dried blood plasma products in the form of UV light or microwaves, or by heating said fluidizing gas.

38. A method according to claim 34, wherein a solvent is added to said blood plasma fractions before said fractions are introduced into said bed of said fluidizing gas to chemically inactivate viruses, thereby eliminating a need for step d).

39. A method according to claim 34, wherein said fluidizing gas is circulated and reconditioned wherein said reconditioning of said fluidizing gas is carried out by means of a condenser for dehumidification and of a heat exchanger for heating.
